(19) United States
(12) Patent Application Publication
(10) Pub. No.: US 2014/0379025 A1
(43) Pub. Date: Dec. 25, 2014

(54) ENTEROATMOSPHERIC FISTULA TREATMENT DEVICES AND METHODS

(71) Applicant: CURASEAL INC., Santa Clara, CA (US)

(72) Inventors: Harold F. CARRISON, Pleasanton, CA (US); Akshay MAVANI, Los Altos, CA (US); Stephen PIERALDI, San Bruno, CA (US)

(73) Assignee: CuraSeal Inc.

(21) Appl. No.: 14/309,205

(22) Filed: Jun. 19, 2014

Related U.S. Application Data
(60) Provisional application No. 61/837,597, filed on Jun. 20, 2013.

(51) Int. Cl. A61B 17/00

(52) U.S. Cl. CPC A61B 17/0057; A61B 2017/00641; A61B 2017/00646
USPC 606/213

(57) ABSTRACT

An implantable device covers and seals an enteroatmospheric fistula or other short-tract opening in tissue and assists in healing the fistula. The implantable device may include a cap that is configured to be positioned on a first side of the tissue and to substantially cover a first opening of the short-tract fistula. The implantable device may also include an anchor member operably connected to the cap. The anchor member may be configured to be positioned on a second side of the tissue and to substantially cover a second opening of the short-tract fistula. Also, the implantable device may include a plug structure operably connected to the cap and the anchor member. The plug structure may be configured to fill the short-tract fistula.

### CROSS-REFERENCE TO RELATED APPLICATION(S)

The present application claims priority to U.S. Provisional Patent Application No. 61/837,597, entitled “Enteroatmospheric Fistula Treatment Devices and Methods,” filed on Jun. 20, 2013. The full disclosure of the above-listed patent application is hereby incorporated by reference herein.

### FIELD

The present disclosure relates generally to medical devices, and more specifically, to treatment devices for enteroatmospheric fistulas.

### BACKGROUND

Fistulas are abnormal tissue-lined pathways or communications between two surfaces of the body. For example, fistulas may develop between body cavities and organs, or between cavities or organs and the surface of the body. A fistula pathway or tract includes a void in the soft tissues extending from a primary fistula opening to a blind ending or leading to one or more secondary fistula openings. Fistulas may develop due to a wound, may be the consequence of infection or abscess formation, or may be purposefully developed (e.g., tracheostomy tracts, gastric feeding tube tracts, etc.). However, most abnormal fistulas may typically occur congenitally, after surgery, from surgery related complications, or from trauma. Fistulas may often have tracts or pathways that are epithelialized, endothelialized, or mucosalized.

Fistulas may form between almost any two organs. For example, fistulas may occur between internal organs and the skin (e.g., enterocutaneous fistulas, gastrocutaneous fistulas, anal fistulas, etc.), or between two internal organs (e.g., gastrointestinal fistulas, colovesicular fistulas, etc.). A perforated intestine or bowel exposed through an open abdominal wound is referred to as an “enteroatmospheric fistula.”

Some fistulas may close on their own and may not cause a person significant harm, while some fistulas are life-threatening and may lead to death. For example, an enterocutaneous fistula between the intestinal tract and the skin can cause intestinal content to enter into the abdomen, which can result in significant medical issues. Further, fistulas are often difficult to treat. For example, while negative pressure may often be used to treat other types of abdominal wounds, in the case of enteroatmospheric fistulas, negative pressure may draw enteric succus from the intestinal tract into the abdomen, which can lead to sepsis. Also, it may be difficult to simply suture or stitch an enteric fistula closed. For example, the tissue may be severely damaged, and adding additional perforations to suture the tissue closed may further damage the tissue, preventing healing.

One method of treating a fistula may be surgery in which the fistula and portions of the affected organs are removed. However, this type of surgery is often a major procedure and the mortality rate may be extremely high. Furthermore, patients undergoing this type of surgery, for example, for an enterocutaneous fistula, may have chronic inflammation near the affected area, and may have dense adhesions and highly friable tissues, further complicating the procedure. Other treatment options may include implantable devices designed to aid in the closure of the fistula by the body itself. However, some of these devices may cause an adverse immunological reaction, may allow leakage of fluid from around the device, may become dislodged, or may migrate from their current position as the patient moves.

The information included in this Background section of the specification, including any references cited herein and any description or discussion thereof, is included for technical reference purposes only and is not to be regarded as subject matter by which the scope of the invention as defined in the claims is to be bound.

### BRIEF SUMMARY

One example of the disclosure may include an implantable device for plugging a short-tract opening in tissue, such as a short-tract fistula, and assisting in healing the short-tract opening. The implantable device may include a cap configured to be positioned on a first side of tissue and to substantially cover a first (e.g., proximal) opening of a fistula tract in the tissue. The implantable device may also include an anchor member operably connected to the cap. The anchor member may be configured to be positioned on a second side of the tissue and to substantially cover a second (e.g., distal) opening of the fistula tract. The implantable device may further include a plug structure operably connected between the cap and the anchor member. The plug structure may be configured to be positioned or received within the fistula tract. A short-tract fistula may comprise a region having a relatively constant diameter, as well as a tapered region. Devices described herein may be configured to accommodate this physiology.

Another example of the disclosure may include a method of treating a fistula or wound. The method may include inserting an anchor member into a fistula (e.g., a short-tract fistula) in tissue. The anchor member may then be manipulated (e.g., rotated and/or expanded) so that a first (e.g., distal) opening of the fistula tract is substantially covered by the anchor member. A cap may then be positioned over a second (e.g., proximal) opening of the fistula tract (e.g., after the anchor member is positioned). The cap and the anchor member may then be operably connected to form a seal over at least one of the first and second openings of the fistula tract.

A further example may include an implantable device for treatment of a fistula (e.g., a short-tract fistula in tissue). The device may include a first member configured to be positioned on a first side of a fistula, covering a first opening of the fistula tract. The device may also include a second member configured to be operably connected to the first member and positioned on a second side of the tissue to cover a second opening of the fistula tract. A plug structure may be operably connected to the first and second members and may be configured to be received within the fistula.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

These and other aspects and embodiments will be described in further detail below, in reference to the attached drawing figures.

### BRIEF DESCRIPTION OF DRAWINGS

FIG. 1A is a front elevational view of a person having an enteroatmospheric fistula (i.e., a perforated intestine accessible through a wound opening in the epidermal layer).

FIG. 1B is an enlarged cross-sectional view of the fistula in the intestine.

FIG. 2 is an isometric view of an implantable device in an extended position configured to be inserted within the enteric fistula of FIG. 1B.

FIG. 3A is a cross-sectional view of the implantable device of FIG. 2 partially inserted within the enteric fistula.

FIG. 3B is a cross-sectional view of the implantable device of FIG. 2 inserted through the enteric fistula.

FIG. 3C is a cross-sectional view of the implantable device of FIG. 2 partially plugging the enteric fistula.

FIG. 3D is a cross-sectional view of the implantable device of FIG. 2 fully inserted within and plugging the enteric fistula.

FIG. 3E is a top plan view of the implantable device of FIG. 2 inserted within the enteric fistula.

FIG. 4A is a cross-sectional view of the implantable device of FIG. 2 inserted in the fistula opening with an anchor member in an insertion configuration.

FIG. 4B is a cross-sectional view of the implantable device of FIG. 2 inserted in the fistula opening with the anchor member in an expanded position.

FIG. 5 is an isometric view of another example of the implantable device of FIG. 2 in a contracted or insertion configuration.

FIG. 6A is a cross-sectional view of the implantable device of FIG. 5 positioned over the fistula opening before insertion.

FIG. 6B is a cross-sectional view of the implantable device of FIG. 5 inserted into the fistula with the anchor member in an insertion configuration.

FIG. 6C is a cross-sectional view of the implantable device of FIG. 5 inserted into the fistula with the anchor member in an expanded configuration.

FIG. 7 is an isometric view of another embodiment of an implantable enteroatmospheric fistula treatment device.

FIG. 8A is a cross-sectional view of the implantable device of FIG. 7 inserted into the fistula.

FIG. 8B is an enlarged cross-sectional view of FIG. 8A illustrating securing members of the implantable device.

FIG. 8C is a top plan view of the implantable device of FIG. 7 inserted into the fistula.

FIG. 9 is an isometric view of another embodiment of an implantable enteroatmospheric fistula treatment device.

FIG. 10A is a cross-sectional view of the implantable device of FIG. 9 inserted into the fistula.

FIG. 10B is a top plan view of the implantable device of FIG. 9 inserted into the fistula.

FIG. 11A is an isometric view of another embodiment of an implantable enteroatmospheric fistula treatment device.

FIG. 11B is an exploded front cross-sectional view of the implantable device of FIG. 11A.

FIG. 12A is a cross-sectional view of the implantable device of FIG. 11A inserted into the fistula.

FIG. 12B is a top plan view of the implantable device of FIG. 11A inserted into the fistula.

FIG. 13 is an exploded view of another example of the implantable device of FIG. 11A.

### DETAILED DESCRIPTION

Embodiments of an implantable device for treating enteroatmospheric fistulas, short-tract fistulas, and other openings in tissue, such as wounds, are described herein. In some embodiments, the implantable device plugs and covers a fistula or wound opening to promote healing (in certain embodiments, without requiring sutures or stitching). The implantable device may be inserted into the fistula or wound opening to close or seal the fistula or wound opening, and may be formed of or include one or more materials that encourage tissue growth in order to help permanently close the fistula or wound opening. The implantable device may be used for a number of different types of fistulas, tissue openings, or other wounds.

The implantable device may include a cap and an anchor member that may be connected together via a plug section or support structure comprising tissue growth enhancing material (e.g., collagen). This configuration may allow tissue to grow into and around the tissue growth enhancing material of the plug section or support structure. Additionally, the cap and the anchor member may be pulled together to seal the tissue opening by either the tissue growth enhancing material plug section or a separate retaining or securing structure (e.g., sutures, bioabsorbable hook and loop, or both). Thus, the implantable device may provide both fistula sealing and a framework for tissue ingrowth.

The first member or cap of the device may be configured to be positioned on a first or exterior surface of the damaged tissue, or against or over a first (e.g., proximal) opening of the short-tract fistula. The cap may, for example, be made of one or more impermeable biocompatible and/or bioabsorbable materials. As such, the cap may be capable of preventing liquids (e.g., enteric succus) and other materials from entering into or exiting out of the fistula opening, while also substantially preventing damage to, and an autoimmune response from, the body as the material may be eventually absorbed by the body. This is important, for example, in the case of an intestinal fistula, where the impermeable cap of the device may prevent intestinal matter and liquids from passing through the fistula into the abdominal cavity. Similarly, the second member or anchor member may be configured to be positioned over a second (e.g., distal) opening of the short-tract fistula (e.g., an opening in an interior surface of the damaged tissue) and may, for example, be made of one or more impermeable biocompatible and bioabsorbable materials. Of course, other appropriate materials may alternatively or additionally be used. The cap and anchor member may be operably connected together, sandwiching the tissue surrounding the fistula therebetween. The cap and the anchor member may be connected together in any appropriate fashion, including but not limited to a plug section, sutures, a keying configuration, hook and loop fasteners, a collagen plug, via magnetic forces, or by other similar structures.

The anchor member may be configured to be inserted into the fistula opening and then expanded and manipulated. This expansion and manipulation may substantially secure the anchor member in place on the second or interior side of the tissue insertion through the opening. In one example, the anchor member may include a series of stacked sheets of uniform or varying diameter that may be folded or compressed while inserted into a first opening of the fistula and then may expand or unfurl when exiting a second opening of the fistula.

In many configurations, the cap and the anchor member may be spaced apart from one another on opposing sides of the tissue surrounding the fistula, but substantially secured in position. This may allow the damaged tissue to grow together within the space between the surfaces of the cap and the anchor, while the fistula is closed or sealed. Additionally, the cap, the anchor member, and/or the separating or securing structure may be bioabsorbable, such that they may eventually be absorbed into the body.

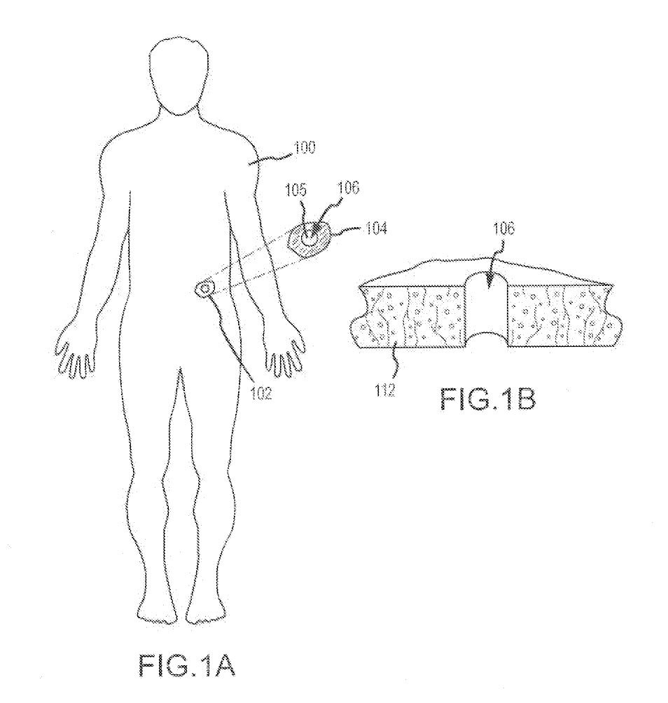

FIG. 1A is a front elevation view of a person 100 who has a perforated intestinal tract 105. An open wound 102 in the abdominal wall 104 exposes a fistula 106 in the intestinal tract 105 to the atmosphere. The fistula 106 may be difficult to treat due to the additional wound 102 in the abdominal cavity 104. FIG. 1B is an enlarged view of the damaged enteric tissue 112 illustrating the fistula 106. The fistula 106 may extend through one or multiple layers of the tissue 112, and may have any of a number of different dimensions and shapes.

FIG. 2 is an isometric view of an implantable device 110 for closing the entire fistula 106. The implantable device 110 extends to cover the margins of the tissue 112 on both sides of the fistula 106 and plugs the fistula tract, thereby providing a framework for growth of the tissue 112 to permanently close the fistula 106. Although the implantable device 110 is illustrated as closing a fistula 106 in the intestinal tract 105, it may be used to close substantially any short length opening in tissue, including but not limited to short-tract fistulas, colon tears, blood vessel perforations, etc.

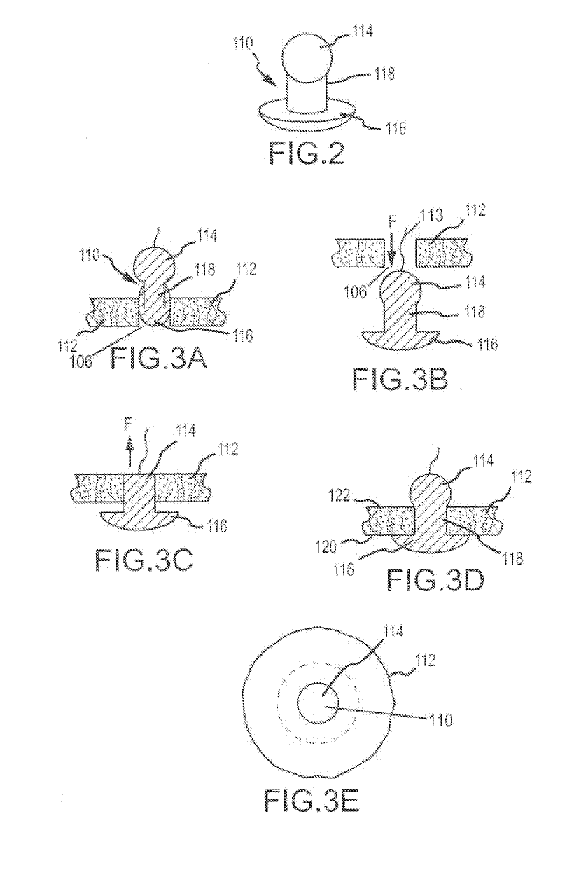

The implantable device 110 is configured to be inserted into the fistula 106 in the tissue 112. FIGS. 3A-3D are cross-sectional views of the implantable device of FIG. 2 partially inserted within the enteric fistula, inserted through the enteric fistula, partially plugging the enteric fistula, and fully inserted within and plugging the enteric fistula, respectively. FIG. 3E is a top plan view of the implantable device of FIG. 2 inserted within the enteric fistula. As shown, the implantable device 110 may include a cap 114, an anchor member 116 operably connected or coupled to the cap 114, and a plug structure 118. The cap 114 may have a smaller diameter than the anchor member 116 when the anchor member 116 is in its expanded position (see, e.g., FIG. 2). The cap 114 is configured to be inserted over a first (e.g., proximal) surface 122 (FIG. 3D) of the tissue 112 and over and partially into the opening 106.

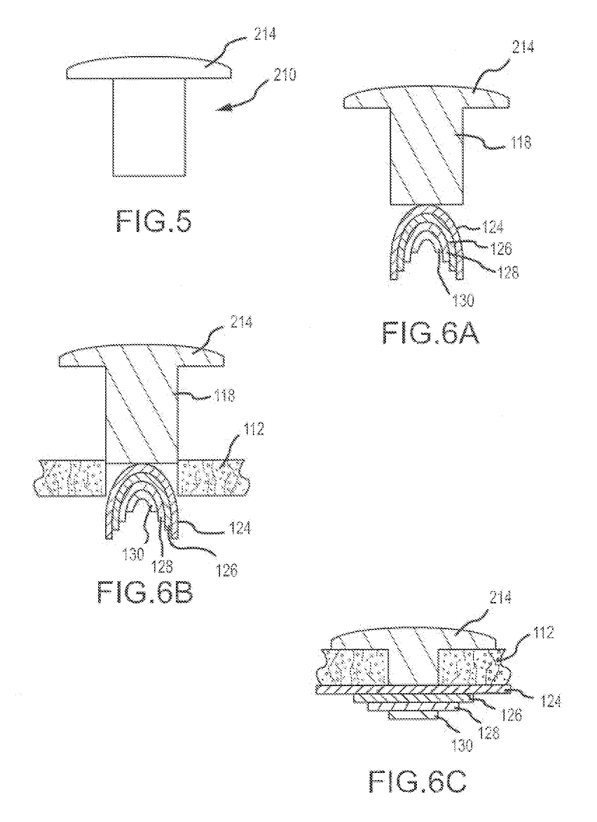

The cap 114 may be formed as a bulbous extension of the plug structure 118 covering one end of the fistula 106. The bulbous cap 114 may be configured to have a larger diameter than a diameter of the opening of the fistula 106, to substantially prevent fluids and other materials from entering or exiting the fistula 106. The cap 114 may be formed of one or more fluid-impermeable materials (e.g., fluid-impermeable silicone), thus preventing fluids and other materials from passing around or through the cap 114 into or out of the fistula 106. In some cases, the cap 114 may be bioabsorbable (e.g., the cap 114 may be formed of one or more bioabsorbable polymers), so that the cap 114 may eventually be absorbed by the body of the person 100. Also, the cap 114 may be other shapes or sizes (see, e.g., FIG. 6A).

The cap 114 may be bulbous and may operably extend downward to form the plug structure 118. Referring to FIGS. 2 and 3D, the plug structure 118 may be a shaft that extends downward (or distally) from a bottom surface of the cap 114 and is operably connected to the anchor member 116. The plug structure 118 operably connects the cap 114 to the anchor member 116 and spaces the two apart from one another. This may advantageously prevent the cap 114 and the anchor member 116 from pinching or otherwise forcing the tissue 112 from its position therebetween. The plug structure 118 may be constructed out of a tissue growth enhancement material, such as collagen. Thus, the plug structure 118 may provide a type of framework or scaffolding through which tissue may grow to fill the fistula 106. Other suitable materials may alternatively or additionally be used.

As shown in FIG. 3A, the anchor member 116 may be substantially flexible, so that it may be inserted into the fistula and may then expand outwards (as shown in FIG. 3D). Referring to FIG. 3B, in one embodiment, the implantable device 110 may be inserted through the fistula, anchor member 116 first, until the entire device 110 is positioned on the opposite side of the tissue. After the implantable device 110 is inserted, the surgeon may pull a pull cord 113 operably connected to the cap 114. Referring now to FIG. 3C, as the cap 114 is forced upwards, the cap 114 and the plug structure 118 may be reinserted into the fistula. The anchor member 116 is not reinserted and forms a seal on a bottom opening of the fistula. Referring now to FIG. 3D, once the cap 114 has reached the other opening of the fistula it may expand to form a plug or seal on the opposite side of the fistula. FIG. 3E is a top plan view of the implantable device 110 inserted within the fistula 106.

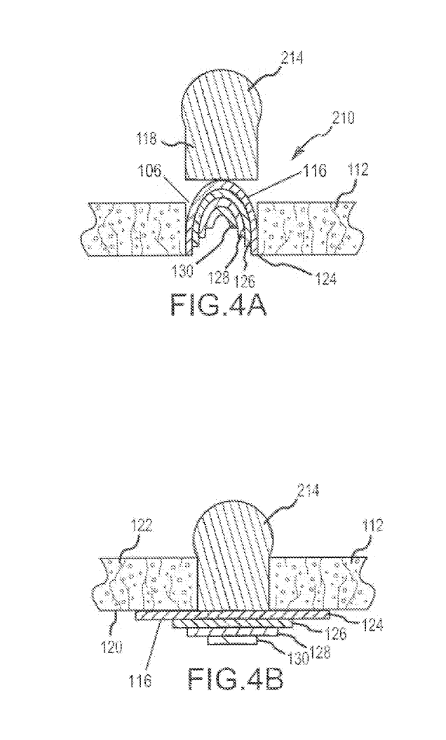

Referring now to FIGS. 4A and 4B, the anchor member 116 may comprise a series of sheets 124, 126, 128, 130 of successively smaller size that may be compressed or deformed to fit through the fistula and may then expand or reform (e.g., to the original sheet form). The sheets 124, 126, 128, 130 may be contracted or folded before the implantable device 110 is inserted into the fistula 106. After the sheets 124, 126, 128, 130 pass through the fistula and enter the enteric cavity, the plug structure 118 is positioned in the fistula 106 and the sheets 124, 126, 128, 130 may expand outwards. Thus, the sheets 124, 126, 128, 130 of the anchor member 116 may cover the second end of the fistula 106 and may be wider than the opening 106, but may still be able to be inserted into the opening 106. The anchor member 116 covers the opening of the fistula 106 on the interior wall of the tissue 112, and acts to anchor the implantable device 110 within the fistula 106, preventing the implantable device 110 from being easily moved or forced from the fistula 106. The sheets 124, 126, 128, 130 may comprise any appropriate material or materials (e.g., collagen), and may comprise the same or different materials.

FIG. 4A is a cross-section of the implantable device 110 inserted into the fistula 106 prior to expansion of the anchor member 116, and FIG. 4B is a cross-sectional view of the implantable device 110 inserted into the fistula 106 after expansion of the anchor member 116. The sheets 124, 126, 128, 130 may be deformed to bend or fold or curve around one another in order to be inserted into the fistula 106. For example, a small diameter inner sheet 130 may be bent or folded downward from a center connection point with an adjacent sheet 128. A middle sheet 128 of larger diameter than the inner sheet 130 may be folded adjacent to and substantially over and around the inner sheet 130, and a second middle sheet 126 of larger diameter than the first middle sheet 128 may similarly be deformed around the first middle sheet 128. An outer sheet 124 of a larger diameter may be folded over the middle sheet 126 and thus over both the middle and inner sheets 128, 130. In some embodiments, the sheets 124, 126, 128, 130 may be connected at a midpoint of each sheet 124, 126, 128, 130.

Referring to FIG. 4B, after the sheets 124, 126, 128, 130 are inserted through the fistula 106 and enter the enteric cavity, the sheets 124, 126, 128, 130 may expand outwards. The outer sheet 124 may be positioned adjacent a second surface 120 of the tissue 112, the second middle sheet 126 may be positioned adjacent the outer sheet 124, the first middle sheet 128 may be positioned adjacent the second middle sheet 126, and the inner sheet 130 may be positioned adjacent the first middle sheet 128. In this configuration, the sheets 124, 126, 128, 130 form a stepped, upside-down pyramid or frustum structure. The outer sheet 124 is configured to expand over the inner opening in the tissue 112, so as to form a cover or seal over the fistula 106. The middle sheets 126, 128 and the inner sheet 130 provide a self-supporting structure to ensure that the span of the largest diameter sheet 124 covers and seals against the opening of the fistula 106. The various sheets 124, 126, 128, 130 may be made of slightly different material formulations to increase in stiffness from the largest diameter sheet 124 to the smallest diameter sheet 130. In this way, the smallest sheets may be better able to support the span of the largest sheet 124. Alternatively, based upon the material forming the sheets 124, 126, 128, 130, the inherent structure of the material may increase the relative stiffness as the dimensions decrease.

The sheets 124, 126, 128, 130 may be resilient and flexible, allowing the sheets 124, 126, 128, 130 to be folded or deformed into an insertion position and then to expand or spring outwards upon emerging from the second end of the fistula 106. This may allow the anchor member 116 to be inserted through the fistula 106 without substantially further damaging the tissue 112 or increasing the diameter or size of the fistula 106.

The sheets 124, 126, 128, 130 may be substantially any size or shape, and may be present in any suitable number, as long as they can contract to pass through the fistula 106. For example, in some embodiments, the anchor member 116 may be formed of a single support sheet that may expand outwards on a pull cord similar to an umbrella. Other appropriate anchor member configurations may also be used.

Referring now to FIGS. 3A and 4B, when the implantable device 110 is positioned in the fistula 106 and the anchor member 116 is expanded, the implantable device 110 may substantially cover the fistula 106 on both surfaces of the tissue 112. The cap 114 is configured to be positioned over a first (e.g., proximal) opening of the fistula 106 within a first surface 122 of the tissue 112. The plug structure 118 is inserted into the fistula 106 and may be configured to have approximately the same diameter as the fistula 106, thus filling the fistula 106. The anchor member 116, when expanded, covers a second (e.g., distal) opening of the fistula 106 and is positioned adjacent a second surface 120 of the tissue 112. The cap 114 and the anchor member 116 may be spaced apart from one another by a distance at least equal to a thickness of the tissue 112. For example, as shown in FIG. 3A, the plug structure 118 may have a length approximately the same as a thickness of the tissue 112, and may thereby provide an appropriate separation distance between the cap 114 and the anchor member 116.

As noted, both the anchor member 116 and the cap 114 may comprise one or more impermeable materials. In this manner, fluids (e.g., enteric succus), waste (e.g., feces), and other materials may be substantially prevented from traveling from the second surface 120 of the tissue 112 to the first surface 122 of the tissue 112 via the fistula 106. Thus, the implantable device 110 may help to prevent medical complications resulting from the escape of fluids, waste, and the like through the fistula.

Also, as the support structure 118 may comprise one or more tissue growth enhancing materials, such as collagen, the implantable device 110 may provide a tissue growth framework. The tissue 112 may be encouraged to grow within the fistula 106 between the cap 114 and the anchor member 116 throughout the framework of the plug structure 118, to fill the fistula 106 and thereby heal the tissue damage. Furthermore, as the cap 114 and anchor member 116 substantially sandwich or encase the tissue 112, lateral tissue growth may be encouraged across the diameter of the ends of the fistula 106.

The implantable device 110 may include a variety of different embodiments. As an example, FIG. 5 is an isometric view of another configuration of the implantable device 210, which may be substantially similar to the implantable device 110 illustrated in FIG. 2. However, the implantable device 210 may include a differently shaped cap 214. In this embodiment the cap 214 is substantially planar or disk-shaped, rather than being bulbous. Other embodiments of implantable devices may comprise caps of still different shapes.

FIG. 6A shows the implantable device 210 prior to insertion into the fistula 106, FIG. 6B shows the implantable device 210 during insertion into the fistula 106, and FIG. 6C shows the implantable device 210 after insertion into the fistula 106. As shown in FIG. 6A, before insertion, the sheets 124, 126, 128, 130 may be deformed to pass through the fistula 106, as explained above with respect to FIG. 4A. As can been seen, the sheets 124, 126, 128, 130 may fold downward to warp around one another. As shown in FIG. 6B, as the plug structure 118 is inserted within the fistula 106, the sheets 124, 126, 128 may begin to expand as they reach the second side of the fistula 106. Now referring to FIG. 6C, once the plug structure 118 is fully inserted into the fistula 106, the sheets 124, 126, 128, 130 may completely expand to form the anchor member 116. The anchor member 116 covers the second end of the fistula 106.

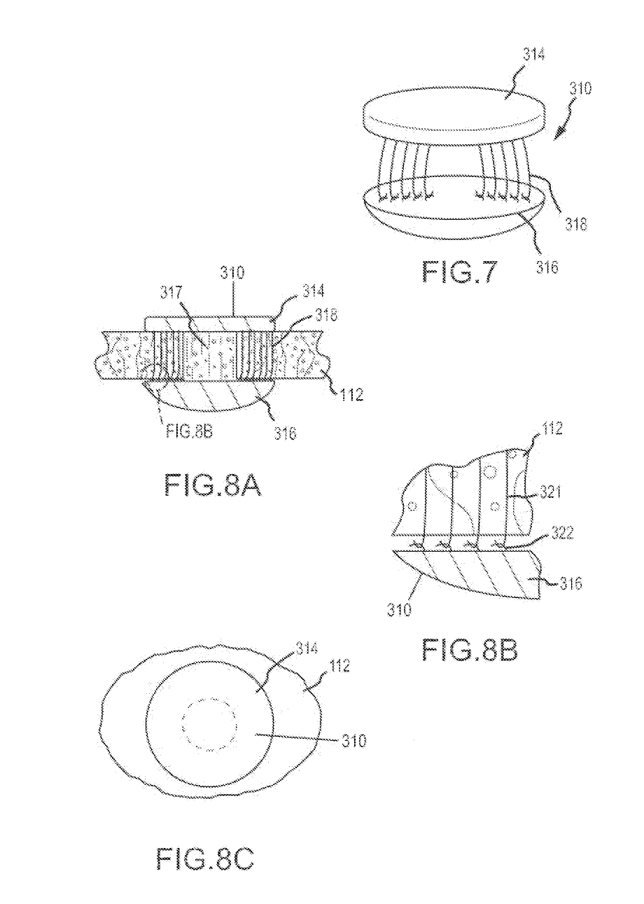

FIG. 7 is an isometric view of another embodiment of the implantable device 310. The implantable device 310 may be substantially similar to the implantable device 110, as it may include a cap 314 and an anchor member 316. However, the implantable device 310 may not include a plug structure. Rather, the anchor member 316 and the cap 314 may be operably connected via securing members 318. Additionally, the anchor member 316 may be an integral member, rather than being formed of multiple sheets as in prior embodiments. Of course, other suitable configurations of anchor members may also be used with securing members.

FIG. 8A is a cross-sectional view of the implantable device 310 inserted within the fistula 106, FIG. 8B is an enlarged view of a portion of FIG. 8A, and FIG. 8C is a top plan view of the implantable device 310 inserted within the fistula 106. The anchor member 316 may be a generally disk-shaped member having a diameter larger than the opening of the fistula 106. In some embodiments, the anchor member 316 may be deformed and inserted through the fistula 106 where, once the anchor member has unfurled in the caving adjacent the second side 120 of the tissue 112, the anchor member may be manipulated by the surgeon. For example, the anchor member 316 may be manipulated for placement adjacent the second side 120 of the tissue 112, and may be positioned to substantially cover the opening of the fistula 106. In such embodiments, the anchor member 316 may be substantially similar to the cap 314 and may be formed of an impermeable material to seal the fistula 106.

FIG. 8B is an enlarged view of the implantable device 310 inserted within the fistula 106 as shown in FIG. 8A. Once the anchor member 316 has been positioned to cover an opening of the fistula 106, the cap 314 and the anchor member 316 may be secured together. Securing members 318 may be operably connected to each of the cap 314 and the anchor member 316. In certain embodiments, securing members 318 that are connected to the cap 314 may also be connected to opposing securing members 318 that are, in turn, connected to the anchor member 316. In some embodiments, the securing members 318 may be inserted into the tissue 112 surrounding the fistula 106.

In an exemplary embodiment, and referring to FIG. 8B, at least some of the securing members 318 may include a hook securing member 321 and a loop securing member 322. The hook securing members 321 may be configured to engage or connect with the loop securing members 322. In one example, the bottom surface of the cap 314 may be formed with the hook securing members 321 and the top surface of the anchor member 316 may be formed with the loop securing members 322. The hook securing members 321 may be pressed through the tissue 112 and exit into the cavity on the opposite side of the fistula 106 to engage the loop securing members 322. Other embodiments and combinations of securing members may also be used.

The securing members 321, 322 function as a hook and loop system in order to secure the cap 314 and the anchor member 316 together, thereby preventing the cap 314 and the anchor member 316 from drifting away from one another. In some embodiments, the hook securing members 321 may be relatively rigid, such that they can pierce the tissue 112 and further act as spacers to space the cap 314 apart from the anchor member 316, to avoid pinching the tissue 112. In certain embodiments, the securing members 318 may be made of one or more bioabsorbable materials so that over time they may eventually be absorbed into the tissue 112.

As shown in FIG. 8A, when inserted, the implantable device 310 sandwiches the tissue 112 between the cap 314 and the anchor member 316. A collagen plug 317 may be placed within the fistula 106 between the cap 314 and the anchor member 316, to promote tissue growth within the fistula 106. This configuration may help to ensure that the ruptured tissue 112 will more quickly heal. Additionally, the cap 314 may be positioned so as to cover the fistula 106, helping to prevent fluid and other materials from entering or exiting the fistula 106.

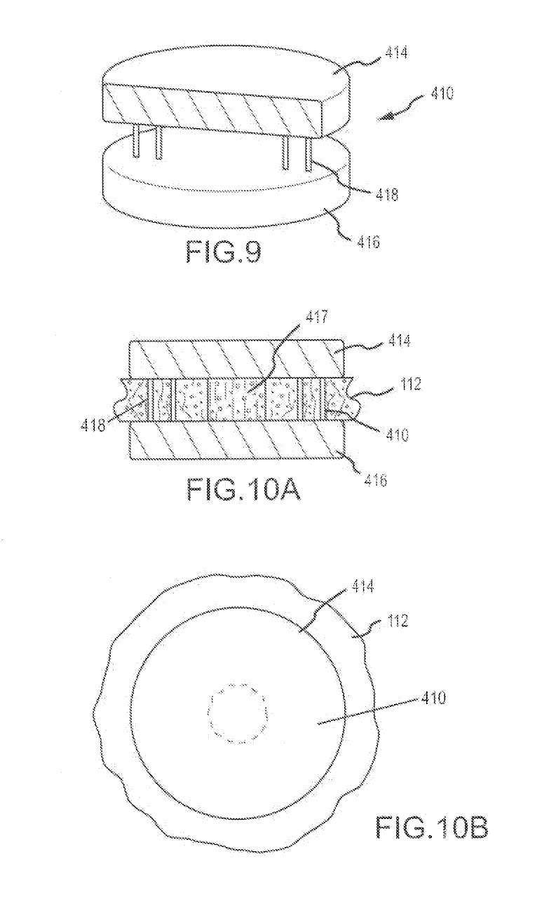

In another embodiment, and referring now to FIG. 9, the implantable device 410 may use an attractive force (e.g., a magnetic force) to secure a cap 414 and an anchor member 416 in place. FIG. 10A is a cross-sectional view of the implantable device 410 inserted within the fistula 106, and FIG. 10B is a top plan view of the implantable device 410. In this embodiment, the cap 414 and the anchor member 416 may have substantially the same dimensions.

In this embodiment of the implantable device 410, both the cap 414 and the anchor member 416 may include one or more types of magnetic materials, such as ferromagnetic materials, dispersed throughout their bodies. Non-limiting examples of ferromagnetic materials include iron, iron oxides, magnetite and ferrofluids. Because of the magnetic material(s), the cap 414 and the anchor member 416 may attract each other across the tissue 112 when properly aligned. This magnetic attraction may function similarly to the previously described securing members 320, in that the magnetic force positions the cap 414 substantially above the anchor member 416.

The anchor member 414 may be deformed for insertion into the fistula 106 and then manipulated within the enteric cavity to recover its normal, flat, disk-like shape and cover the opening. As the anchor member 416 may include magnetic elements dispersed through its body, a surgeon may use a magnetic tool to manipulate the anchor member 416 after it has been inserted through the fistula 106. The positioning magnet may align the anchor member 416 so that it substantially covers the fistula 106.

With continuing reference to FIGS. 9 and 10A, the implantable device 410 may additionally include spacer members 418 located between the cap 414 and the anchor member 416. The spacer members 418 may help to prevent a direct connection between the cap 414 and the anchor member 416, which could pinch or force the tissue 112 out from between the cap 414 and the anchor member 416. In some cases, the spacer members 418 may be used if the magnetic attraction between the cap 414 and the anchor member 416 causes these two components to compress over time and necrotize the tissue 112 around the margins of the fistula 106, thereby enlarging the fistula 106 rather than promoting healing. In certain embodiments, the spacer members 418 may be substantially the same height as a thickness of the tissue 112. This may allow the cap 414 and the anchor member 416 to be spaced far enough apart to avoid placing too much pressure on the tissue 112, but close enough together to provide a good seal with respect to the tissue 112.

The spacer members 418 may have any appropriate configuration, and in some embodiments may be substantially cylindrical shafts or rods that are inserted into the tissue 112 between the cap 414 and the anchor member 416. The spacer members 418 may, for example, comprise one or more bioabsorbable materials (e.g., that are relatively rigid). Non-limiting examples of bioabsorbable materials which may be appropriate include bioabsorbable polymers, such as poly-L-lactic acid (PLLA), polyglycolic acid (PGA), poly(DL-lactide/glycolide) copolymer (PDLA), and polycaprolactone (PCL). In one embodiment, a spacer member 418 may be formed on and extend from a surface of the cap 414.

In operation, the anchor member 416 may be inserted into the fistula 106 and manipulated within the enteric cavity to cover the hole and adjacent margins of the tissue 112. Once the anchor member 416 is positioned, the spacer members 418 may be inserted into the tissue 112 and operably connected to the anchor member 416. Alternatively or additionally, spacer members 418 may be inserted into the fistula 106, to connect to the anchor member 416 without passing through the tissue 112. The cap 414 may then be placed on top of the first surface of the tissue 112, substantially over the fistula 106 and the anchor member 416. Alternatively, the spacer members 418 may be formed on and extend from a surface of the cap 414. The cap 414 may be placed on the tissue 112 and the spacer members 418 may be pushed through the tissue 112. In some cases, some of the spacer members 418 may be pushed through the tissue 112, while other of the spacer members 418 may be advanced through the fistula 106 without entering the tissue 112. In certain embodiments, all of the spacer members 418 may be advanced through the fistula 106 without entering the tissue 112. The attractive force of the cap 414 and the anchor member 416 may act to hold each of them in place over the first and second ends of the fistula 106.

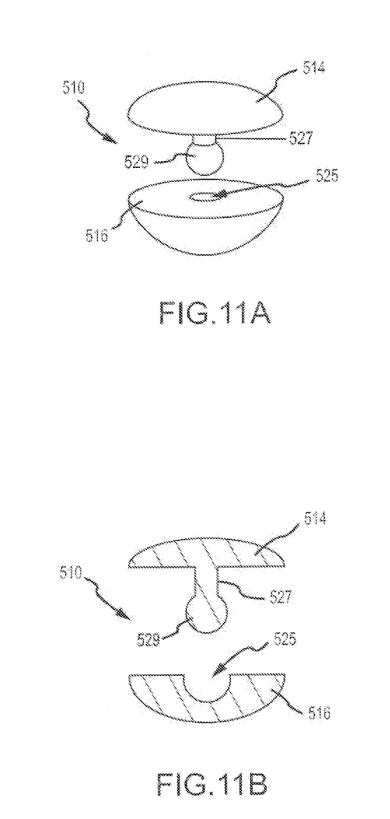

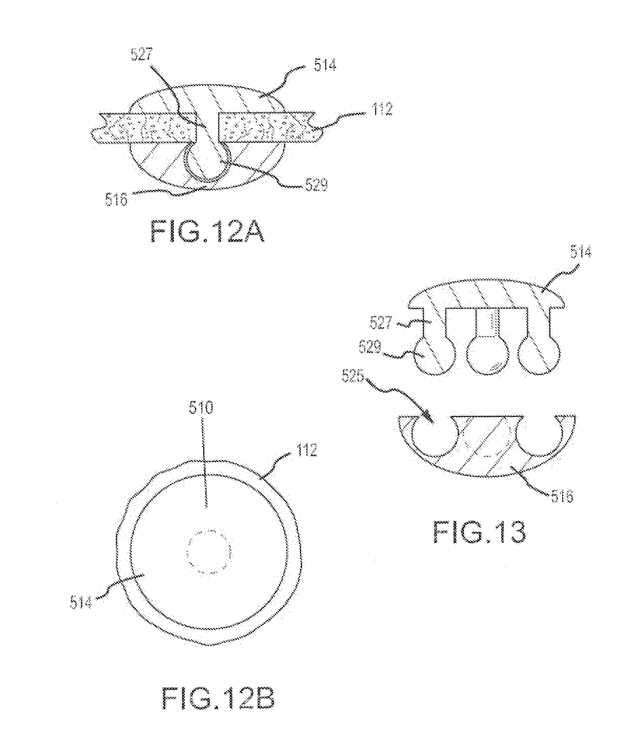

FIGS. 11A and 11B are an exploded isometric view and an exploded front elevational view, respectively, of another embodiment of the implantable device 510. In this embodiment, the implantable device 510 may include a cap 514 having a stem 527 with a head 529 at a first end. The implantable device 510 may also include an anchor member 516 defining a receiving cavity 525 configured to receive the head 529 of the cap 514. FIG. 12A is a cross-sectional view of the implantable device 510 inserted within the fistula 106, and FIG. 12B is a top plan view of the implantable device 510 inserted within the fistula 106. The cap 514 and the anchor member 516 are configured to be secured together in a snap-fit or other suitable fastening configuration.

The cap 514 may have a generally mushroom-shaped top portion that extends downward at a middle portion to form the stem 527. In other embodiments, a stem may not be centrally located on a cap, or multiple stems may be employed. The stem 527 may be similar to the plug structure 118 illustrated in FIG. 2A, in that the stem 527 is configured to be inserted into the fistula 106. A first end of the stem 527 that is inserted into the fistula 106 may extend outward to form a bulbous head 529. The stem 527 and the head 529 may each be formed of a tissue growth enhancer, such as collagen. This may allow for tissue 112 to grow around and through the stem 527 and the head 529 to fill the fistula 106.

The anchor member 516 may be shaped substantially the same as the cap 514. As discussed above, the anchor member 516 may define a receiving cavity 525 for receiving the head 529 of the cap 514. Positioning the head 529 within the receiving cavity 525 operably connects the cap 514 to the anchor member 516. The receiving cavity 525 may be substantially the same size as the head 529 and may receive the head 529 in a snap-fit connection, thereby securing the cap 514 to the anchor member 516.

In some embodiments, the cap 514 and the anchor member 516 may include multiple stems and receiving cavities (e.g., for securely connecting a large diameter implantable device 510 to cover and fill a larger diameter fistula). For example, FIG. 13 shows the cap 514 having three stems 527 extending from a bottom surface and the anchor member 516 including three receiving cavities 525 for receiving each head 529. Also, although a rounded head and cavity configuration is illustrated, other forms of keyed structures are possible, and the snap-fit connection illustrated in FIGS. 11A and 13 is just one exemplary embodiment.

In operation, the anchor member 516 may be deformed (e.g., folded or rolled) and inserted into the fistula 106. The anchor member 516 may then be manipulated within the enteric cavity so that it is unfolded or unrolled and moved into position. Next, the operator may align the cap 514 with the fistula 106, so that the stem 527 and the head 529 may be inserted into the fistula 106. The head 529 may then be inserted past the second surface 120 of the tissue 112, and received into the receiving cavity 525 so that the head 529 is secured within the cavity 525. In some cases, the anchor member may comprise a pulling member that may be used to hold the anchor member in position while the cap is pushed down. Once the cap 514 and the anchor member 516 are engaged, the tissue 112 may be substantially sandwiched between them. The length of the stem 527 may provide an appropriate setoff distance between the cap 514 and anchor member 516, such that the tissue 112 is not substantially compressed and compromised.

The foregoing description has broad application. For example, while embodiments disclosed herein may focus on closing enteroatmospheric fistulas or other short-tract fistulas, the concepts disclosed herein may equally apply to closing other type of wounds and tissue openings. Similarly, although fistulas and wounds may be discussed with respect to humans, the devices and techniques disclosed herein are equally applicable to other animals. Accordingly, the discussion of any embodiment is meant only to be exemplary and is not intended to suggest that the scope of the disclosure, including the claims, is limited to these examples.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. An implantable device configured to be inserted into a short-tract fistula in tissue, the implantable device comprising:
a cap configured to be positioned on a first side of the tissue and to substantially cover a first opening of the short-tract fistula;
an anchor member operably connected to the cap and configured to be positioned on a second side of the tissue and to substantially cover a second opening of the short-tract fistula; and
a plug structure operably connected between the cap and the anchor member and configured to be positioned within the short-tract fistula.

2. The implantable device of claim 1, wherein the first opening is a proximal opening of the short-tract fistula.

3. The implantable device of claim 2, wherein the second opening is a distal opening of the short-tract fistula.

4. The implantable device of claim 1, wherein the plug structure comprises collagen.

5. The implantable device of claim 1 further comprising:
a hook securing member operably connected to a surface of the cap; and
a loop securing member operably connected to a surface of the anchor member,
wherein the hook securing member is configured to pass through the tissue and to be inserted into the loop securing member.

6. The implantable device of claim 1, wherein:
the cap further comprises a first magnetic material; and
the anchor member comprises a second magnetic material substantially attracted to the first magnetic material.

7. The implantable device of claim 1, wherein the anchor member comprises a first sheet having a first configuration allowing for passage through the short-tract fistula and a second expanded configuration having a dimension larger than a diameter of the short-tract fistula.

8. The implantable device of claim 7, wherein the anchor member further comprises a second sheet operably connected to the first sheet and having a third configuration and an expanded fourth configuration having a dimension larger than a diameter of the short-tract fistula but smaller than the dimension of the first sheet.

9. The implantable device of claim 1, wherein the plug structure is directly connected to the cap and is connected to the anchor member via a snap-fit connection.

10. The implantable device of claim 9, wherein the plug structure further comprises:
a stem extending from a bottom surface of the cap; and
a head extending from a bottom end of the stem.

11. The implantable device of claim 10, wherein the anchor member further comprises a cavity configured to receive at least a portion of the head.

12. A method of treating a wound, the method comprising:
inserting an anchor member into a short-tract fistula in tissue;
manipulating the anchor member so that a first opening of the short-tract fistula is substantially covered by the anchor member;
positioning a cap over a second opening of the short-tract fistula; and
operably connecting the cap and the anchor member to form a seal over at least one of the first and second openings of the short-tract fistula.

13. The method of claim 12, wherein operably connecting the cap and the anchor member comprises:
inserting a plug structure into the short-tract fistula; and
receiving at least a portion of the plug structure in a cavity of the anchor member.

14. The method of claim 12, wherein operably connecting the cap and the anchor member comprises:
inserting a hook securing member operably connected to a bottom surface of the cap through the tissue; and
connecting the hook securing member through a loop securing member operably connected to or integral with the anchor member.

15. The method of claim 12, further comprising inserting spacer members into the tissue between the cap and the anchor member.

16. The method of claim 12, wherein the cap comprises a first magnetic element and the anchor member comprises a second magnetic element.

17. The method of claim 16, wherein manipulating the anchor member comprises:
positioning a third magnetic element substantially over the anchor member; and
displacing the third magnetic element to move the anchor member.

18. An implantable device, comprising:
a first member configured to be positioned on a first side of a tissue to cover a first opening of a short-tract fistula in the tissue;
a second member configured to be operably connected to the first member and positioned on a second side of the tissue to cover a second opening of the short-tract fistula; and
a plug structure operably connected to the first and second members and configured to be received within the short-tract fistula.

19. The implantable device of claim 18, wherein the second member is configured to be deformed into a first configuration before being inserted into the short-tract fistula and expanded into a second configuration after being inserted into the short-tract fistula.

20. The implantable device of claim 19, wherein the second member comprises a first sheet configured to be deformed into the first configuration and expanded into the second configuration to have a dimension larger than a diameter of the short-tract fistula.

21. The implantable device of claim 18, wherein the plug structure is directly connected to the first member and connected via a snap fit connection to the second member.

22. The implantable device of claim 18, wherein the plug structure is a tissue growth framework material.
